(19) United States
(12) Patent Application Publication
(10) Pub. No.: US 2026/0283962 A1
(43) Pub. Date: Sep. 24, 2026

(54) PHARMACEUTICAL COMPOSITION AND METHOD FOR ENHANCING SOLUBILITY OF POORLY SOLUBLE ACTIVE PHARMACEUTICAL INGREDIENTS

(71) Applicant: MERCK PATENT GMBH, Darmstadt (DE)

(72) Inventors: Thomas KIPPING, Darmstadt (DE); Jonas LINDH, Uppsala (SE); Julian QUODBACH, Uppsala (SE)

(73) Assignee: MERCK PATENT GMBH, Darmstadt (DE)

(21) Appl. No.: 18/881,106

(22) PCT Filed: Jul. 3, 2023
(86) PCT No.: PCT/EP2023/068253

§ 371 Date: Jan. 3, 2025

(30) Foreign Application Priority Data
Jul. 6, 2022 (EP) 22183236.3

(51) Int. Cl. A61K 9/20

(52) U.S. Cl. CPC A61K 9/2095; A61K 9/2031

(57) ABSTRACT

The present disclosure generally relates to the use of poloxamer in additive manufacturing technologies and techniques. More specifically, the present disclosure relates to the use of poloxamer in a selective laser sintering (SLS) method to additively manufacture an object, in particular a pharmaceutical dosage form. Also described is a process for selective laser sintering a sinter powder to form a pharmaceutical dosage form, in which the sinter powder includes at least one active pharmaceutical ingredient and at least one poloxamer with a melting point of 20° C. or higher.

### TECHNICAL FIELD

The present disclosure generally relates to the use of poloxamer in additive manufacturing technologies and techniques. More specifically, the present disclosure relates to the use of poloxamer in a selective laser sintering (SLS) method to additively manufacture an object, in particular a pharmaceutical dosage form.

### BACKGROUND

Poloxamers are non-ionic poly (ethylene oxide) (PEO)-poly (propylene oxide) (PPO) copolymers. They have many functions in pharmaceutical formulations as surfactants, emulsifying agents, solubilizing agent, or dispersing agents. All poloxamers have similar chemical structures but with different molecular weights and composition of the hydrophilic PEO block (a) and hydrophobic PPO block (b) (Bodratti A M, Alexandridis P. Formulation of poloxamers for drug delivery. Journal of functional biomaterials. 2018; 9(1):11).

Poloxamers are can be used to improve the oral bioavailability of low solule compounds by heating solid dispersions. The preparation of the amorphous solid dispersion with poloxamers is usually quite complex. One strategy is to dissolve polymer and drug substance and perform a freeze drying step to create the final ASD (Song C K, Yoon I-S, Kim D-D. Poloxamer-based solid dispersions for oral delivery of docetaxel: Differential effects of F68 and P85 on oral docetaxel bioavailability. International Journal of Pharmaceutics. 2016; 507(1):102-8). Also many ternary systems are described in literature where small amounts of poloxamers are added to a polymeric carrier in order to enhance the bioavailability of low soluble compounds (Vasconcelos T, Prezotti F, Araújo F, Lopes C, Loureiro A, Marques S, et al. Third-generation solid dispersion combining Soluplus and poloxamer 407 enhances the oral bioavailability of resveratrol. International Journal of Pharmaceutics. 2021:595:120245).

Selective Laser Sintering (SLS) is one of the most popular additive manufacturing processes that creates a three-dimensional (3D) object layer-by-layer. The process apples layers of powder material on top of each other sequentially, where each layer of powder is sintered or coalesced together with a laser according to the computer aided drawing (CAD) geometry of the part.

SLS is a powder bed based additive manufacturing technique to produce complex three-dimensional parts. In SLS, a rasterized laser is used to scan over a bed of polymer powder, sintering it to form solid shapes in a layer-wise fashion. When the laser beam scans the powder, the powder melts due to the rising temperature, and layer by layer, the final part approaches full density and should result in properties of the bulk material (that is, the polymer). By controlling the energy input it is possible to control the density of the sintered material and to achieve parts ranging from highly porous to almost full dense. In theory, every thermoplastic polymer that can be transformed into a powder form can be processed via this technique, but the reality is that every material behaves differently, often unpredictably, during melting, coalescence, and consolidation, and often requires unique SLS processing parameters. The bed temperature and laser energy input, for example, can be selected based on the processing window of the polymer's thermal profile as well as its energy absorption. Laser parameters can also be selected based on the powder's particle size and shape.

There are different types of polymer particles that are generally used in the SLS process. Semi-crystalline resins such as polyamides including PA12, PA11, and PA6, polylactic acid (PLA), polyether ether ketone (PEEK), polyethylene (PE), polypropylene (PP), and others are used. The most common polymer powder employed is polyamide PA12. The common name for polyamide is nylon. For example polyamide PA12 is also known as nylon 12, polyamide PA6 is also known as nylon 6. A layer-upon-layer structure is formed by sintering the polymer particles together with a laser above the melting point of the polymer according to the CAD geometry file of the part.

For the use of poloxamer as pharmaceutical excipient in selective laser sintering there is currently comparably little to no experience. So far, the suitability of different poloxamer grades is unknown.

There is a need for suitable polymers as pharmaceutical excipients having improved properties for the method of selective laser sintering. In particular, the laser sintering of the polymer should yield pharmaceutical dosage forms, wherein the API is dispersed in the polymer forming an amorphous solid dispersion. Polymers for that purpose should show an improved amorphization of the API and/or a beneficial API release. Furthermore, there is a need for polymers that stabilize the API or that can be beneficially used in the method of SLS to prevent the API from degradation.

### SUMMARY OF THE INVENTION

Surprisingly it was found that certain poloxamers can be beneficially used in a process for selective laser sintering of sinter powder to form a pharmaceutical dosage form. Poloxamers with a melting point of 20° C. or higher are particularly suitable for selective laser sintering.

Unexpectedly, it was shown that low process temperatures are needed with the poloxamers of the present invention to successfully manufacture an amorphous solid dispersion. The low temperatures have several advantages compared to alternative polymers, e.g. re-usability of the poloxamer after a printing circle, increase of printing speed, accessibility of the method for a broader range of SLS printers and a reduction in energy needed for the print. Certain poloxamers show an surprisingly fast API release.

Additionally, there is an optimum range of melting point (Tm), molecular weight (MW) and weight percentage of ethylene oxide (EO) chains (Weight EO %) which leads to pharmaceutical dosage form with the beneficial properties as mentioned above.

In a further embodiment of the invention, the poloxamer has a melting point between 20° C. and 60° C. and/or an average molecular weight of 3000 Da or higher and/or a weight percentage of ethylene oxide chains between 50% and 90%. In another embodiment the poloxamer is poloxamer P188 or P407.

In a further embodiment of the invention, the poloxamer has a melting point between 40° C. and 55° C. and/or an average molecular weight between 7000 Da and 12000 Da and/or a weight percentage of ethylene oxide chains between 75% and 85%. In another embodiment the poloxamer is poloxamer P188.

In another aspect, the invention provides a process for for producing a pharmaceutical dosage form by selective laser sintering of sinter powder, comprising the steps of
(a) providing a sinter powder comprising at least one active pharmaceutical ingredient and at least one poloxamer with a melting point of 20° C. or higher and
(b) operating a selective laser sintering apparatus that selectively fuses layers of the sinter powder to produce the pharmaceutical dosage form.

A further aspect of the invention concerns a sinter powder for selective laser sintering, comprising at least one active pharmaceutical ingredient and at least one poloxamer with a melting point of 20° C. or higher.

A further aspect of the invention concerns a pharmaceutical dosage form obtainable by a process as mentioned above, in particular a pharmaceutical dosage form produced by selective laser sintering of sinter powder, wherein the sinter powder comprises at least one active pharmaceutical ingredient and at least one poloxamer with a melting point of 20° C. or higher.

### DETAILED DESCRIPTION OF THE INVENTION

An embodiment of the invention is the use of poloxamer in a process for selective laser sintering of sinter powder to form a pharmaceutical dosage form, wherein the sinter powder comprises at least one active pharmaceutical ingredient and poloxamer.

According to the invention selective laser sintering is a process in which a laser beam is used to sinter and/or melt a powder bed filled with a powder mixture containing polymer by scanning the laser according to the cross-section of a digital model. A polymer version of the digital model is produced in a layer-by-layer fashion by laser scanning successive layers of powder mixture.

The process will require a selective laser sintering printer equipped with a laser source and a galvanometric system for scanning the laser on the powder bed surface or, alternatively, a xy motion system where the actual laser source is moved to scan the powder bed. The printer must also provide a powder application system to spread the powder in layers as well as some heating capabilities to heat the build chamber and the surface of the powder bed.

According to the invention, the powder mixture used is prepared by mixing poloxamer powder, excipients and API. (Excipients: pigment, colloidal silica). The poloxamer powder is first sieved through a 300 micron sieve and the material which pass through the sieve are mixed with excipients and API and mixed in a turbula mixer for 30 min. The resulting mixture is then sieved through a 300 micron sieve and the material which pass through the sieve is loaded into the printer.

After loading the mixture, the printer is preheated to a set temperature below the Tg of the poloxamer and the printing process is initiated. In the printing process parameters such as chamber and print bed temperature are set to appropriate values obtained via experimental studies to provide printed tablets with desirable properties with respect to mechanical and morphological properties. Other parameters influencing the process are laser energy input and layer height of each applied layer. The laser energy input can be controlled in a number of ways depending on which type of printer is used and usually via adjusting laser scanning speed, hatching space (distance between scanned laser lines) or by adjusting the energy output by the laser.

Once the printing process is finalized the printed tablets are allowed to slowly cool down in the printer before being removed and cleaned from surrounding, unsintered powder.

Poloxamers are amphiphilic polymers, with two hydrophilic blocks and a hydrophobic block in the middle. A poloxamer is a polyethylene glycol (PEG)/polypropylene glycol (PPG) tri-block copolymer whereby one PPG block is flanked on both sides with a PEG block. The polyethylene glycol (PEG) part is often also called polyethylene oxide (PEO) part. The polypropylene glycol (PPG) part is often also called the polypropylene oxide (PPO) part.

Poloxamer grades are commonly named with the letter P (for poloxamer) followed by three digits that is officially used by USP and EP. It describes the composition of the polymer as follows: the first two digits multiplied by 100 represents the molecular weight of the PO block and the last digit multiplied by 10 provides the percentage of EO in %.

Poloxamer P188 in average is composed of 80% EO, while the remaining 20% PO make up for 1800 g/mol. Poloxamer P407 is a poloxamer with an average polyoxypropylene molecular mass of 4000 g/mol and a 70% polyoxyethylene content.

Poloxamers have the general formula (I)

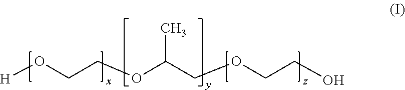

For different poloxamers numbers of x (PEO), y (PPO chain) and z (PEO) are varying over a broad range, depending on the type of poloxamer. For poloxamer P188 the PPO chain contains in the average a unit number ranging from 25 to 30, and each PEO is composed of 75 to 85 EO units in average, with a molecular weight ranging from 7680 to 9510 Da. For poloxamer P407 the PPO chain contains in the average a unit number of 56, and each PEO is composed of approximately 101 EO units in average, with an molecular weight ranging from 9840 to 14600 Da.

Poloxamer 407 (a=101, b=56) with molecular weight ranging from 9840 to 14600 Da.

Table 1 is showing types of poloxamers monographed in the European Pharmacopoeia (Ph. Eur.) and United States Pharmacopeia (USP).

TABLE 1

| | Average | | EO units | PO units |
|---|---|---|---|---|
| | Molecular | Weight | (each flank | Ph. Eur. |
| | Weight | EO | Ph. Eur. | (range); |
| Poloxamer | (USP/EP) | [%] USP | (range); USP | USP |
| 124 | 2090-2360 | 46.7 ± 1.9 | 10-15; 12 | 18-23; 20 |
| 188 | 7680-9510 | 81.8 ± 1.9 | 75-85; 80 | 25-30; 27 |
| 237 | 6840-8830 | 72.4 ± 1.9 | 60-68; 64 | 35-40; 37 |
| 338 | 12,700-17,400 | 83.1 ± 1.7 | 137-146; 141 | 42-47; 44 |
| 407 | 9840-14,600 | 73.2 ± 1.7 | 95-105; 101 | 54-60; 56 |

Table 2 is showing the melting point, molecular weight and weight percentage of ethylene oxide chains (Weight EO %) of the most common poloxamners as published in Russo, Villa. Poloxamer Hydrogels for Biomedical Applications. Pharmaceutics. 2019; 11:671.

TABLE 2

| | Weight | Average Molecular | Melting |
|---|---|---|---|
| Poloxamer | EO [%] | Weight [Da] | Point [° C.] |
| P105 | 50 | 1900 | 7 |
| P108 | 80 | 4700 | 48 |
| P122 | 20 | 1630 | −26 |
| P123 | 30 | 1850 | −1 |
| P124 | 40 | 2200 | 16 |
| P182 | 20 | 2500 | −4 |
| P183 | 30 | 2650 | 10 |
| P184 | 40 | 2900 | 16 |
| P185 | 50 | 3400 | 27 |
| P188 | 80 | 8400 | 52 |
| P212 | 20 | 2750 | −7 |
| P215 | 50 | 4150 | 27 |
| P217 | 70 | 6600 | 48 |
| P234 | 40 | 4200 | 34 |
| P235 | 50 | 4600 | 34 |
| P237 | 70 | 7700 | 49 |
| P238 | 80 | 11,400 | 54 |
| P288 | 80 | 13,000 | 58 |
| P333 | 30 | 4950 | 30 |
| P334 | 40 | 5900 | 32 |
| P335 | 50 | 6500 | 35 |
| P338 | 80 | 14,600 | 57 |
| P402 | 20 | 5000 | 20 |
| P403 | 30 | 5750 | 31 |
| P407 | 70 | 12,600 | 56 |

Some poloxamners are commercially available at different ratios of EO and PO units and in different forms like liquids, pastes and wax-like solids, e.g. Synperonic® (Croda International PLC), Pluronic® (BASF SE), Lutrol® (BASF SE renamed in Kolliphor® and Kollisolv®) or Poloxamer 188 EMPROVE® EXPERT.

Alternatively, poloxamers can be made from raw materials according to methods known in the art (see, for example, U.S. Pat. Nos. 3,579,465 and 3,740,421).

Further information about poloxamers can be found in Hagers Handbuch der Pharmazeutischen Praxis, volume 9 “Stoffe P-Z”, 1994, pages 282 to 284 or Russo, Villa. Poloxamer Hydrogels for Biomedical Applications. Pharmaceutics. 2019; 11:671.

The use of specific poloxamer grades according to the invention is of interest for the formulation of solid oral pharmaceutical dosage forms with an instant, immediate or prolonged API release. An instant or immediate release is preferred.

Poloxamers according to the invention have a melting point of 20° C. or higher.

In a further embodiment, poloxamers according to the invention have a melting point between 20° C. and 60° C., between 30° C. and 60° C., between 40° C. and 60° C. or between 50° C. and 60° C. In a further embodiment, poloxamers according to the invention have an average molecular weight of 3000 Da or higher, between 4000 and 15000 Da or between 7000 and 13000 Da. In a further embodiment, poloxamers according to the invention have a weight percentage of ethylene oxide chains between 50% and 90% or 70% to 85%. In another embodiment the poloxamer is poloxamer P188 or P407.

In a further embodiment, poloxamers according to the invention have a melting point between 20° C. and 60° C., an average molecular weight of 3000 Da or higher and a weight percentage of ethylene oxide chains between 50% and 90%.

In a further embodiment, poloxamers according to the invention have a melting point between 30° C. and 60° C. an average molecular weight between 4000 and 15000 Da and a weight percentage of ethylene oxide chains between 70% to 85%.

In a further embodiment, poloxamers according to the invention have a melting point between 50° C. and 60° C. an average molecular weight between 7000 and 13000 Da and a weight percentage of ethylene oxide chains between 70% to 85%.

In a further embodiment, poloxamers according to the invention have a melting point between 20° C. and 55° C., between 30° C. and 55° C., between 40° C. and 55° C. or between 50° C. and 55° C. In a further embodiment, poloxamers according to the invention have an average molecular weight between 4000 and 12000 Da or between 7000 and 12000 Da. In a further embodiment, poloxamers according to the invention have a weight percentage of ethylene oxide chains between 75% and 85% or 80% to 85%. In another embodiment the poloxamer is poloxamer P188.

In a further embodiment, poloxamers according to the invention have a melting point between 20° C. and 55° C., an average molecular weight between 4000 and 12000 Da and a weight percentage of ethylene oxide chains between 75% and 85%.

In a further embodiment, poloxamers according to the invention have a melting point between 40° C. and 55° C., an average molecular weight between 7000 and 12000 Da and a weight percentage of ethylene oxide chains between 75% and 85%.

In a further embodiment, poloxamers according to the invention have a melting point between 50° C. and 55° C., an average molecular weight between 7000 and 12000 Da and a weight percentage of ethylene oxide chains between 80% to 85%.

In a further embodiment, the poloxamer according to the invention has a melting point of approximately 52° C. and/or an average molecular weight between 7680 and 9510 Da and/or a weight percentage of ethylene oxide chains between 80% to 85%. In a further embodiment of the invention, the poloxamer is poloxamer P188.

In a further embodiment, the poloxamer according to the invention has a melting point of approximately 56° C. and/or an average molecular weight between 9840 and 14600 Da and/or a weight percentage of ethylene oxide chains between 70% to 75%. In a further embodiment of the invention, the poloxamer is poloxamer P407.

The above mentioned embodiments poloxamers, poloxamer specification and poloxamer grades apply equally for the use of poloxamers in a process for selective laser sintering of sinter powder, the process for producing a pharmaceutical dosage form by selective laser sintering of sinter powder, the sinter powder for selective laser sintering and the pharmaceutical dosage form produced by selective laser sintering of sinter powder.

A further embodiment of the invention is a sinter powder for selective laser sintering, comprising at least one active pharmaceutical ingredient and at least one poloxamer according to the invention.

For the avoidance of doubt, the sinter powder according to the invention include sinter powders with any of the poloxamers, poloxamer specifications or poloxamer grades as defined above.

In a further embodiment of the invention, the sinter powder and the pharmaceutical dosage form may comprise further pharmaceutically acceptable excipients. Pharmaceutically acceptable excipients comprise flow control agents, such as silicon dioxide, fillers, plasticizers, surfactants, light-absorbing material, such as ruby red or Candurin pigments and other suitable components that are well known to those skilled in the art.

Depending on the wavelength of the light emitted by the laser, a light-absorbing material (pigment) which absorbs light at the wavelength emitted may be required. These light-absorbing materials can contain transition metals for absorption at around 450 nm or carbon for a wider range covering the visible- and near IR range. Light absorption is a process by which light is absorbed and converted into energy. When light is absorbed heat is generated. So the selective absorption of light by a particular material occurs because the frequency of the light wave matches the frequency at which electrons in the atoms of that material vibrate.

Light-absorbing materials are all materials suitable for the SLS method as described above and known to the skilled person in the art. Preferably light-absorbing materials which have been demonstrated to work at 445 nm laser irradiation are used, e.g. Candurin NXT, Ruby Red, Candurin Gold Sheen, Aluminum Lake, activated carbon (also works at 808 nm) or iron oxide (Fe2O3). More preferably ruby red is used.

Carbon dioxide laser emitting at around 10 microns will usually not require addition of light-absorbing materials as C—H bonds absorb energy well at this wavelength and this type of bonds can be found in most polymers.

For the avoidance of doubt, flow control agents, plasticizers, surfactants or other suitable components are not needed for the beneficial properties according to the invention, e.g. stabilizing the amorphous form of the APIs. Yet those components can be used for other purposes, e.g. to optimize the process of manufacturing of the pharmaceutical composition or oral dosage form according to the invention.

Furthermore, the pharmaceutical composition according to the invention may comprise additional pharmaceutically acceptable hydrophilic or lipophilic polymers.

In a preferred embodiment the sinter powder further comprises alight-absorbing material.

As used herein, the phrase “pharmaceutically acceptable” refers to all excipients, polymers, compounds, solvents, dispersion media, flow control agents, carriers, coatings, active agents, isotonic and absorption delaying agents, and the like that do not produce an allergic or similar untoward reaction when administered to humans in general. The use of such material in pharmaceutical compositions is well known in the art.

In a preferred embodiment the particle size of the sinter powder has a D50 of 200 μm or lower. Preferably, the particle size (D50) of the sinter powder is between 20 μm and 200 μm, 20 μm and 150 μm or 20 μm and 100 μm.

In a preferred embodiment the particle size of the poloxamer has a D50 of 200 μm or lower. Preferably, the particle size (D50) of the poloxamer is between 20 μm and 200 μm, 20 μm and 150 μm or 20 μm and 100 μm.

A further embodiment of the invention is a process for producing a pharmaceutical dosage form by selective laser sintering of sinter powder, comprising the step of
(a) providing a sinter powder comprising at least one active pharmaceutical ingredient and at least one poloxamer with a melting point of 20° C. or higher and
(b) operating a selective laser sintering apparatus that selectively fuses layers of the sinter powder to produce the pharmaceutical dosage form.

For the avoidance of doubt, the processes according to the invention include selective laser sintering with any of the poloxamers, poloxamer specifications or poloxamer grades as defined above.

A further embodiment of the invention is a pharmaceutical dosage form obtainable by the process for producing a pharmaceutical dosage form by selective laser sintering of sinter powder as described above.

In a further embodiment the pharmaceutical dosage form is produced by selective laser sintering of sinter powder wherein the sinter powder comprises at least one active pharmaceutical ingredient and at least one poloxamer with a melting point of 20° C. or higher.

For the avoidance of doubt, the pharmaceutical dosage forms according to the invention include pharmaceutical dosage forms with any of the poloxamers, poloxamer specifications or poloxamer grades as defined above.

The API is a biologically active agent. The API may be a small molecule in form of a weak base, a weak acid or a neutral molecule and may be in the form of one or more pharmaceutically acceptable salts, esters, derivatives, analogues, prodrugs, and solvates thereof. The sinter powder and the pharmaceutical dosage form may comprise more than one API. In one embodiment the API is poorly soluble or a lipophilic API.

As used herein, the terms “poorly soluble API”, “poorly water-soluble API” and “lipophilic API” refer to an API having a solubility such that the highest therapeutic dose of the particular API to be administered to an individual cannot be dissolved in 250 ml of aqueous media ranging in pH from 1 to 8 following the definition of low solubility according to the Biopharmaceutics Classification System (BCS) classes 2 and 4. Poorly soluble APIs with weakly basic or weakly acidic characteristics have a pH-dependent solubility profile and can have a wide range of solubility in the aqueous environment of the gastrointestinal tract. APIs falling under BCS classes 2 or 4, respectively, are well known to persons skilled in the art.

In one embodiment the API is a weakly basic API. As used herein, the term “weakly basic API” refers to a basic active pharmaceutical ingredient (API) wherein the basic API does not completely ionize in water.

The at least one active pharmaceutical ingredient (API) according to the invention may be dispersed in the poloxamer forming an amorphous solid dispersion.

A further embodiment of the invention is the use of poloxamer in a process for selective laser sintering of sinter powder to form a pharmaceutical dosage form as described above, wherein an amorphous solid dispersion of at least one active pharmaceutical ingredient in the poloxamer is formed.

As used herein, the term “amorphous solid dispersion” is a dispersion of at least one amorphous API in a polymer matrix. Preferably, the amorphous API is distributed in a molecularly dispersed state within the polymer matrix. In this case, the solid dispersion is a solid solution. Upon dissolution, formulations comprising an amorphous solid dispersion can reach higher solubilities in aqueous media than the crystalline API.

The API included in the pharmaceutical dosage form of the present invention has a sufficient amount to be therapeutically effective. For a given API, therapeutically effective amounts are generally known or readily accessible by persons skilled in the art. Typically, the API may be present in the pharmaceutical dosage form in a weight ratio of API to poloxamer of 0.1:99.1 to 60:40, preferably 1:99 to 50:50, more preferably 5:95 to 40:60 and most preferably 10:90 to 30:70.

It was found that poloxamers can be beneficially used in a process for selective laser sintering of sinter powder to form a pharmaceutical dosage form.

Surprisingly low process temperatures are needed for the selective laser sintering process. Example 1 surprisingly shows that the inventive SLS printing technology with the above mentioned poloxamers was able to successfully manufacture an amorphous solid dispersion with a print bed temperature of approximately 50° C. and a chamber temperature at room temperature. It was not expected that a pharmaceutical dosage form with an amorphous API stabilized in a polymer matrix could be manufactures with the SLS technology at temperatures in that range.

The low temperature have numerous advantages compared to alternative SLS processes with different polymers:
1. Due to the low temperatures, the remaining sinter powder on the print bed after a print circle can be re-used several times without any deterioration in print quality. This has not been seen for any other polymer.
2. The low printing temperatures make it possible to print parts without a heated print chamber which allows for use of simpler and lower cost printers
3. The low printing temperatures make it possible to use a fast printing speed and thereby a reduced production time. Additionally the thermical stress for the API is reduced with a higher printing speed.
4. The low printing temperatures of the polymers reduces the amount of energy necessary for the printing process. Thus, the SLS printing process with the above mentioned poloxamers has a better environmental sustainability.

Event though the process was carried out at temperatures far below the melting point of the model drug substances the crystalline drug was successfully converted in its amorphous form and an amorphous solid dispersion could be manufactured.

Certain poloxamers show a surprisingly fast drug release compared to other poloxamers. In particular, poloxamers having a melting point between 40° C. and 55° C., preferably between 50° C. to 55° C. and/or an average molecular weight between 7000 Da and 12000 Da, preferably between 7000 Da and 9000 Da and/or a weight percentage of ethylene oxide chains between 75% and 85%, preferably between 75% and 80% show a fast API dissolution of the pharmaceutical dosage form compared to poloxamers falling outside the specifications. In another embodiment Poloxamer P188 shows a comparably fast API dissolution. The fast drug dissolution surprisingly is a result of the combination of the process for producing the pharmaceutical dosage form (SLS technology) and the choice of the poloxamer. This has been confirmed by using different 3D printing technologies with the identical poloxamer grades which resulted in no improvement in dissolution of the API.

Certain poloxamers show a surprisingly improved amorphization of the API compared to other poloxamers. In particular, poloxamers having a melting point between 40° C. and 55° C., preferably between 50° C. to 55° C. and/or an average molecular weight between 7000 Da and 12000 Da, preferably between 7000 Da and 9000 Da and/or a weight percentage of ethylene oxide chains between 75% and 85%, preferably between 75% and 80% show a fast API dissolution of the pharmaceutical dosage form compared to poloxamers falling outside the specifications. In another embodiment Poloxamer P188 shows an improved amorphization of the API compared to other poloxamers.

Furthermore, poloxamers having the above-identified melting points, molecular weights and/or weight percentages of ethylene oxide chains assure and stabilize the release and supersaturation of APIs, in particular poorly soluble APIs, in aqueous media thereby preventing crystallization and phase separation. Since a low water solubility of an API in general accompanies a low bioavailability after its administration in a pharmaceutical preparation, the compositions according to the invention also contribute to improving the bioavailability of poorly water-soluble APIs, and particularly weakly basic APIs

As used herein, “bioavailability” is a term meaning the degree to which an API becomes available to the target tissue after being administered to the body of a patient.

After dissolving pharmaceutical dosage forms of the present invention an improved supersaturation is observed and the API is kept better in solution. Furthermore, the API can be better incorporated into the poloxamer matrix during the sintering process

Having entered the gastrointestinal tract, the pharmaceutical dosage form swells and disintegrates in the aqueous environment of the gastrointestinal fluids thereby releasing the API. While a salt form of a weakly basic API may show improved initial aqueous concentration in the acidic gastric fluid, the weakly basic API rapidly converts to the free base form in the more neutral intestinal fluid where the free base-form of the API has a significantly lower equilibrium concentration. Poloxamers according to the invention maintain enhanced concentrations of the API in model solutions simulating acidic and neutral gastrointestinal solutions as compared to poloxamers falling outside the mentioned specifications. Therefore, the pharmaceutical dosage forms according to the invention have the potential to provide enhanced bioavailability of poor solubility APIs. The solubility-improved form of the API in the presence of a poloxamer according to the invention provides a concentration of the API in gastric fluid or simulated gastric fluid that is greater than the concentration of the API provided in the presence of a poloxamer falling outside the mentioned specifications.

### EXAMPLES

### Example 1: SLS Printing

### Preparation of Powder Formulation

API-loaded powder formulations were prepared according to Table 3. All powder mixtures were sieved using a 315 μm stainless-steel test sieve (VWR International AB, Sweden) and mixed using a Turbula shaker (Turbula T2F shaker, Glen Mills, Inc., Clifton, NJ, US) for 15 min. Candurin Ruby Red and colloidal silica (Aerosil) were added to the formulations in order to enhance the laser energy absorption of the powders and to improve powder flowability during the layer application process, respectively. As poloxamer, P188 (Poloxamer 188 EMPROVE® EXPERT, Merck) and P407 (Merck) were used. The formulations were prepared in large enough batches (approx. 1500 mL) to partially fill the build volume (100×100×100 mm) of a Sintratec Kit SLS 3D printer (Sintratec, Brugg, Switzerland).

TABLE 3
Composition of the prepared powder formulations

| Formulation | Poloxamer | Pigment | Flow control | API |
|---|---|---|---|---|
| No. | [wt. %] | [wt. %] | agent [wt. %] | [wt. %] |
| P188-I | P188 | Ruby Red [1] | Aerosil [0.5] | Indomethacin |
| | [88.5] | | | [10] |
| P188-K | P188 | Ruby Red [1] | Aerosil [1] | Ketoconazole |
| | [90.5] | | | [7.5] |
| P407-I | P407 | Ruby Red [1] | Aerosil [0.5] | Indomethacin |
| | [88.5] | | | [10] |
| P407-K | P407 | Ruby Red [1] | Aerosil [1] | Ketoconazole |
| | [90.5] | | | [7.5] |

### Selective Laser Sintering 3D Printing of Dosage Forms

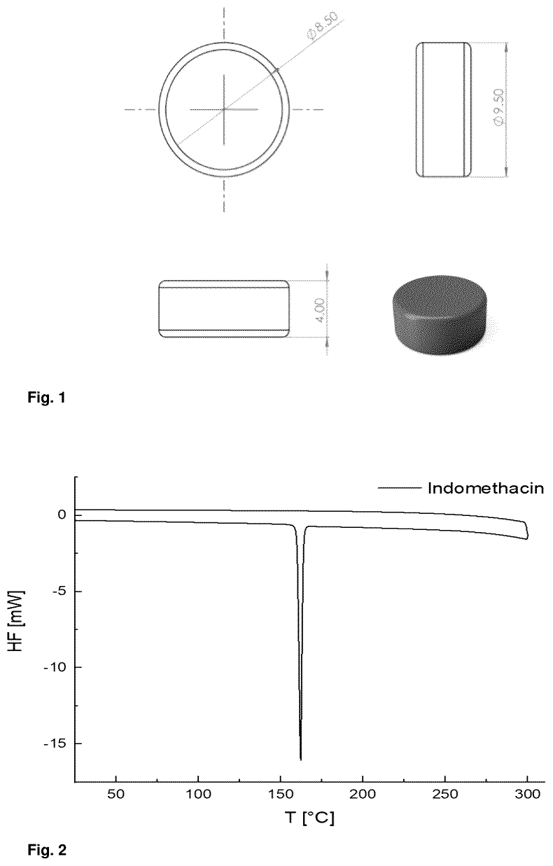

Tablet templates were created and designed in Solidworks 2019 SP05 (FIG. 1), and the obtained standard triangle language file (STL-file) was subsequently prepared for printing in Sintratec software using the process parameters presented in Table 4. The energy density was calculated according the following equation:

$$e = α \frac{4 P}{π \cdot HS \cdot V} = α \frac{4 \cdot 2.3}{π \cdot 0.05 \cdot 200} = α \cdot 0.29 J / {mm}^{2}$$

P—laser power (2.3 W); HS—hatching space (0.05 mm); V—scanning speed (mm/s); α—absorptivity of the powder (coefficient should be measured/calculated for each powder type with respect to absorption at 445 nm).

The 3D printing process was further carried out as follows: The prepared powder formulations (Table 3) were placed and packed into the powder reservoir platform (100×100×100 mm) of the SLS 3D printer. A thin layer of the formulation was thereafter spread onto the build platform after which the powder beds were slowly heated to the temperatures specified in Table 4. The sintering process was carried out using a 2.3 W laser diode (λ=445 nm) in accordance with the models given in the STL-file in a layer-by-layer fashion. A total of 36 tablets were printed per batch, flat to the build platform, using a layer height specified in Table 4. Specific values for the laser scanning speed were chosen when printing the different batches. The finished batches were collected from the build platform at the end of the printing process by sieving. The tablets were additionally de-dusted using pressurized air in order to remove excess powder and stored in sealed containers for further analysis.

FIG. 1 is showing a schematic drawing and 3D model of a tablet (4×9.5 mm). Print speed, print bed temperature and hatching space were adjusted to have the correct amount of energy applied to the materials in order to take into account the different materials having different melting temperatures and different particle sizes which requires different temperatures to sinter properly.

TABLE 4
Process parameters used for each formulation

| | | | | | | | Energy |
|---|---|---|---|---|---|---|---|
| | | Printing | Hatching | | Print bed | Chamber | density |
| Batch | Formulation | speed | space | N of | temperature | temperature | α |
| No. | No | (mm/s) | (μm) | perimeter | (° C.) | (° C.) | (J/mm²) |
| 1 | P188-I | 530 | 125 | 3 | 50 | rt | 0.044 |
| 2 | P188-I | 530 | 100 | 3 | 50 | rt | 0.055 |
| 3 | P188-K | 640 | 125 | 2 | 50 | rt | 0.037 |
| 4 | P188-K | 640 | 100 | 2 | 50 | rt | 0.046 |
| 5 | P407-I | 620 | 125 | 3 | 50 | rt | 0.038 |
| 6 | P407-I | 620 | 100 | 3 | 50 | rt | 0.047 |
| 7 | P407-K | 720 | 125 | 2 | 52.2 | rt | 0.033 |
| 8 | P407-K | 740 | 125 | 2 | 52.1 | rt | 0.032 |

### Characterization of Power Formulations and 3D Printed Dosage Forms

Powder X-ray diffraction (PXRD) diffractograms of the pristine and heat-treated powder formulations as well as the printed dosage forms were collected on a Bruker D8 Advance TwinTwin diffractometer (Bremen, Germany) using Cu-Kα_{1,2} (λ=1.5418 Å) radiation. The instrument was operated at 40 mA and 40 kV, using a step-size of 0.02′ and a data collection time of 1 h.

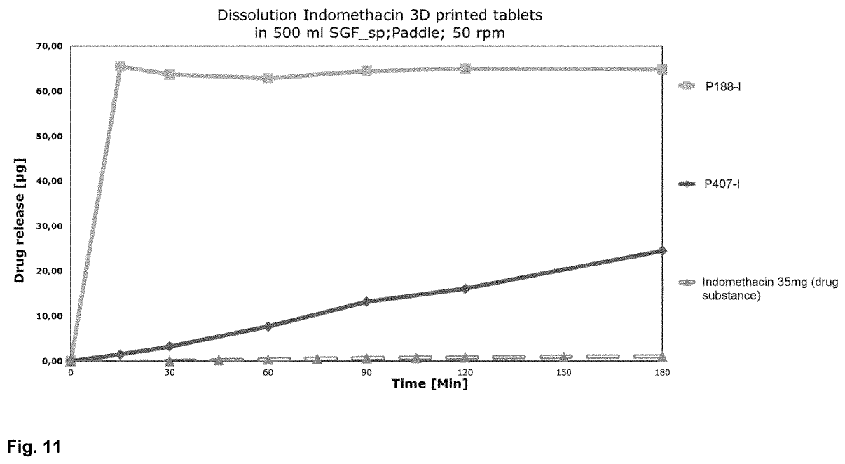

FIGS. 9 and 10 shows diffractograms for indomethacin loaded tablets and ketoconazole loaded tablets. X-ray diffractograms confirm the amorphization of the drug substance within the 3D-printed tablet matrices.

Differential scanning calorimetry (DSC) thermograms were obtained on a Mettler Toledo DSC 3+ (Schwerzenbach, Switzerland) using a heating and cooling rate of 10° C. min⁻¹ and nitrogen as purge gas. Repeated heating-cooling measurements were carried out from −40 to 200° C. and from 200 to 10° C. in the first cycle, and from 10 to 200° C. in the following cycles. The dimensions (n=10) and weights (n=30) of the printed tablet were examined using a digital caliper and an analytical balance (Mettler Toledo XS 64 Analytical Balance, Schwerzenbach, Switzerland).

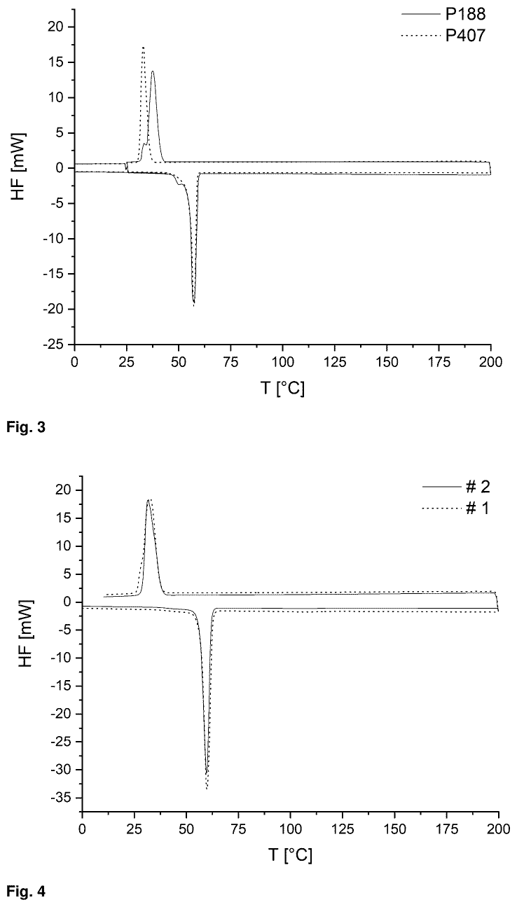

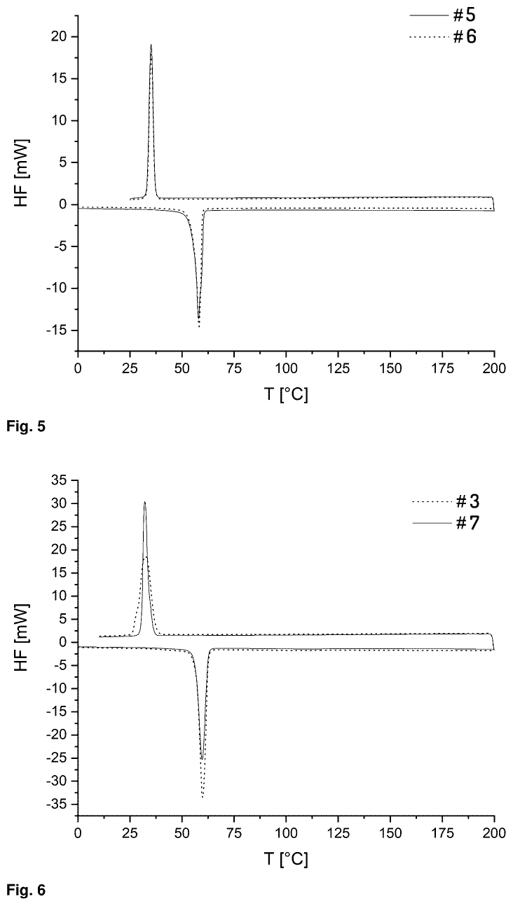

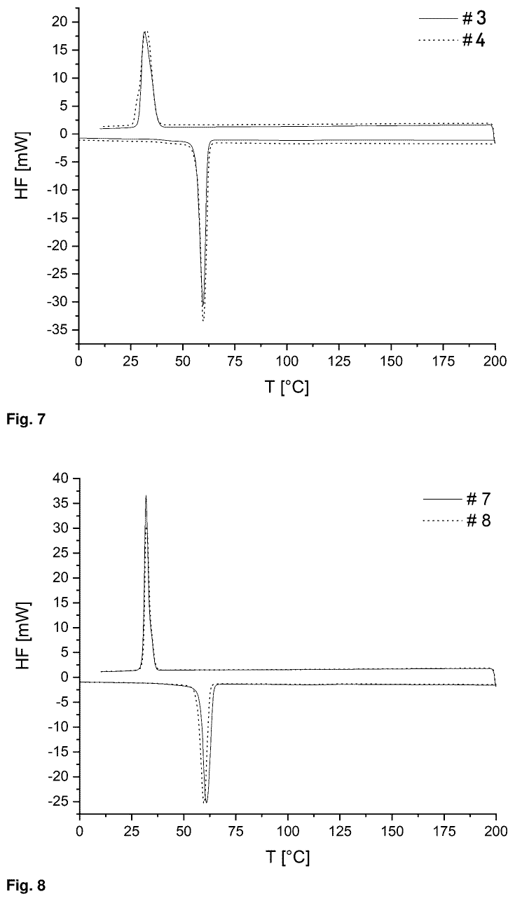

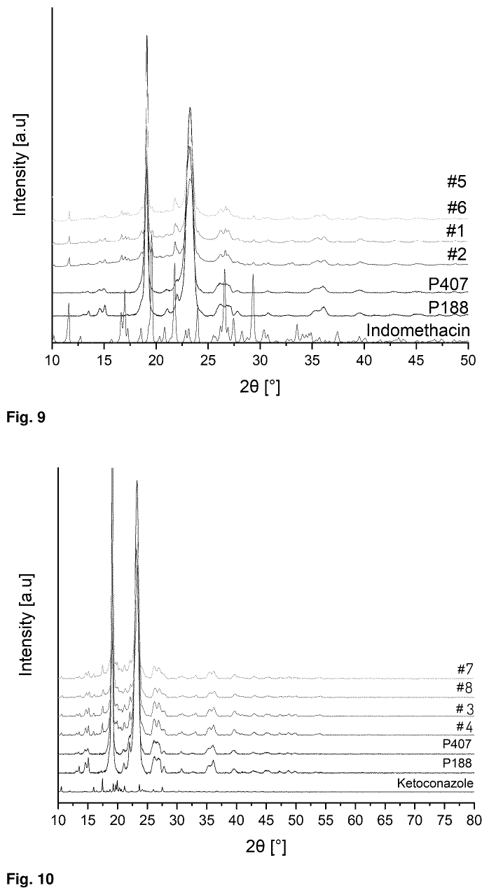

FIGS. 2 to 8 show DSC thermograms for pure indomethacin (FIG. 2), pure polymers/placebo tablets without API (FIG. 3), indomethacin loaded P188 (FIG. 4), indomethacin loaded P407 (FIG. 5), ketoconazole loaded P188 and P407 (FIG. 6), ketoconazole loaded P188 (FIG. 7) and ketoconazole loaded P407 (FIG. 8). The Batch No. (Table 3) are indicated as #No. in the figures. Thermograms of indomethacin indicating a distinct melting peak of the drug substance. It can be shown that the melting peak of indomethacin is not anymore available in the 3D-printed tablets indicating a full amorphization of the drug substance.

Friability tests were carried out in accordance with the European Pharmacopoeia (Ed. 10.0) on approx. 6.5 g of tablets using a Pharmatest PTF E friabilator (Hainberg, Germany) at 25 rpm and for 100 rotations. The tablets were carefully weighed pre- and post-measurement and total weight loss of the tablets (i.e. friability) calculated.

Dissolution data were performed on Sotax AT7 dissolution tester via HPLC from Agilent 1260 infinity II. Mobil phase contains 1000 ml Acetonitril+1000 ml buffer USP (0.01M NaH₂PO*H₂O 1.38 g/L+0.01M Na₂HPO₄ 1.41 g/L) mix 1:1 mix both and ultrasonic for 15 min. Prepare suitable 5-point calibration of Indomethacin in mobil phase.

Dissolution was performed with n=3 tablets in 500 ml SGF_sp, Paddle, 50 rpm for three hours with sampling points at (15, 30, 60, 90, 120 und 180 minutes). 2 ml sample was filtered over 0.45 μm PTFE syringe filters and measured directly with HPLC at 254 nm, 10 μl injection volume with a flow rate 1 ml/min and 5 minutes runtime on LC-18, 30 cm×4 mm, 5 μm column.

Hardness tests were carried out on 10 cylindrical tablets (10 mm in diameter) from each batch in order to define the tablet hardness (given in Newtons, N). A Pharmatest PTB 311E tablet hardness testing instrument (Hainberg, Germany) was used.

The splitting tensile strength was calculated according to the following equation for the cylinder shape:

$$T = \frac{2 P}{π L D}$$

(P—maximum applied load (N), L—length (m), D—diameter)

The friability test was carried out according to EP 5.0, paragraph 2.9.7—Friability of uncoated tablets.

For tablets weighing up to 0.65 g each, take a sample of twenty tablets; for tablets weighing more than 0.65 g each, take ten tablets. Place the tablets on a sieve no. 1000 and remove any loose dust with the aid of air pressure or a soft brush. Accurately weigh the tablet sample and place the tablets in the drum. Rotate the drum 100 times and remove the tablets. Remove any loose dust from the tablets as before. If no tablets are cracked, split or broken, weigh the tablets to the nearest milligram.

TABLE 5
Characterization of 3D printed dosage forms

| Batch | Hardness | Tensile strength | mₚᵣₑ₋ₜₑₛₜ | mₚₒₛₜ₋ₜₑₛₜ | Friability |
|---|---|---|---|---|---|
| No. | (N) | (kPa) | (g) | (g) | (wt. %) |
| 1 | 15 ± 4 | 221 ± 42 | 6.5805 | 6.5446 | 0.55 |
| 2 | 26 ± 6 | 399 ± 92 | 6.6554 | 6.6153 | 0.60 |
| 3 | 26 ± 5 | 412 ± 79 | 6.8569 | 6.828 | 0.43 |
| 4 | 58 ± 10 | 968 ± 173 | 6.5263 | 6.500 | 0.40 |
| 5 | — | — | 6.7889 | 6.7747 | 0.21 |
| 6 | 27 ± 3 | 365 ± 171 | 6.8515 | 6.8447 | 0.10 |
| 7 | 26 ± 5 | 404 ± 78 | 6.8173 | 6.7922 | 0.37 |
| 8 | 26 ± 5 | 420 ± 81 | 6.5142 | 6.4281 | 1.31 |

1. A process for selective laser sintering of sinter powder to form a pharmaceutical dosage form, comprising sintering a sinter powder comprising at least one active pharmaceutical ingredient and at least one poloxamer with a melting point of 20° C. or higher.

2. The process according to claim 1 wherein the melting point of the at least one poloxamer is between 40° C. and 60° C.

3. The process according to claim 1, wherein the average molecular weight of the at least one poloxamer is 3000 Da or higher.

4. The process according to claim 1, wherein the at least one poloxamer has a weight percentage of ethylene oxide chains between 50% and 90%.

5. The process according to claim 1, wherein the at least one poloxamer is poloxamer P188 or P407.

6. The process according to claim 1, wherein the sinter powder further comprises at least one light-absorbing material.

7. A process for producing a pharmaceutical dosage form by selective laser sintering of sinter powder, comprising:
(a) providing a sinter powder comprising at least one active pharmaceutical ingredient and at least one poloxamer with a melting point of 20° C. or higher, and
(b) operating a selective laser sintering apparatus that selectively fuses layers of the sinter powder to produce the pharmaceutical dosage form.

8. The process according to claim 7, wherein the pharmaceutical dosage is an amorphous solid dispersion of the at least one active pharmaceutical ingredient in the at least one poloxamer.

9. The process according to claim 7, wherein the at least one poloxamer is poloxamer P188 or P407.

10. The process according to claim 7, wherein the process temperatures are below the melting point of the at least one poloxamer.

11. The process according to claim 7, wherein the particle size of the sinter powder has a D50 of 200 μm or lower.

12. The process according to claim 7, wherein the sinter powder further comprises at least one light-absorbing material.

13. A sinter powder for selective laser sintering, comprising at least one active pharmaceutical ingredient and at least one poloxamer having a melting point of 20° C. or higher.

14. A sinter powder according to claim 13, further comprising at least one light-absorbing material.

15. A pharmaceutical dosage form obtainable by a process according to claim 7.
